(19) United States
(12) Patent Application Publication
(10) Pub. No.: US 2011/0060195 A1
(43) Pub. Date: Mar. 10, 2011

(54) COSMETIC TREATMENT METHOD FOR TREATING THE SCALP

(75) Inventors: Segolene DE NORAY, Paris (FR); Gabin Vic, Semoy (FR); Eric Parris, Clichy (FR)

(73) Assignee: L'OREAL, Paris (FR)

(21) Appl. No.: 12/855,404

(22) Filed: Aug. 12, 2010

Related U.S. Application Data
(60) Provisional application No. 61/237,719, filed on Aug. 28, 2009.

(30) Foreign Application Priority Data
Aug. 13, 2009 (FR) 09 55671

(51) Int. Cl. A61M 35/00; A61K 8/02; A61K 9/14; A61P 17/14; A61Q 5/00; A61Q 7/00; A61F 7/00; A45D 24/10; A61B 1/06

(52) U.S. Cl. 600/249; 424/401; 424/489; 604/310; 604/291; 132/148

(57) ABSTRACT

Embodiments of the present invention relate to a cosmetic treatment method that treats the human scalp, including applying a cosmetic composition and/or a gas to the scalp with a spray device operating with a compressed gas, a mean size of the sprayed particles of composition lying in the range 10 μm to 35 μm.

### CROSS-REFERENCE TO RELATED APPLICATIONS

This application claims priority under 35 U.S.C. §119(e) that claims the benefit of U.S. Provisional Application No. 61/237,719, filed Aug. 28, 2009, and claims priority under 35 U.S.C. §119 from French Application No. 09 55671, filed Aug. 13, 2009, the entire contents of which are herein incorporated by reference.

### BACKGROUND OF THE INVENTION

1. Field of the Invention

The present invention relates to methods and systems for treating the human scalp.

2. Discussion of Background

It is known to apply a composition to the scalp by pouring it directly onto the head by means of a cannula. By way of example, the composition may be a cleansing, soothing, anti-dandruff, or anti-hairloss composition, or a composition that seeks to stimulate hair regrowth.

The use of devices making it possible to pour the composition directly onto the scalp is not entirely satisfactory, since it is difficult to control accurately the quantity of composition poured, and there exists a risk of running that may lead to a loss of composition and running into the eyes.

In addition, the distribution of the composition over the scalp might not be as uniform as desired, which might result in having to distribute it with the fingers, and might require gloves to be worn, and that could turn out to be problematic when the scalp is sensitive to rubbing or the hair is fragile when rubbed.

It is also known to use airbrushes for applying makeup compositions to the skin, which airbrushes spray the composition in the form of fine droplets.

Application GB 2 287 480 A discloses the use of an airbrush for applying a neutralizing composition to the hair when creating a permanent wave.

### SUMMARY OF THE INVENTION

To the knowledge of the Applicant, airbrushes and other similar devices that operate with compressed gas and in particular with compressed air are not used for applying a cosmetic composition to the scalp in order to treat it.

There exists a need to improve still further treatments for the scalp.

In a first of its aspects, the invention thus provides a cosmetic treatment method for treating the human scalp, wherein a cosmetic composition and/or a gas is applied to the scalp by means of a device that operates with a compressed gas.

The composition is preferably other than a composition for coloring the hair or for shaping the hair or a care product for the hair.

The composition may be a composition for treating the scalp, selected in particular from: anti-dandruff, anti-hairloss, hair-regrowth, anti-seborrheic, anti-inflammatory, anti-irritant or soothing, concealer, stimulating, and protective compositions for the scalp, the composition possibly being cosmetic or dermatological.

The composition is sprayed in the form of droplets and not in the form of foam.

When the composition is an anti-hairloss composition or a composition for stimulating hair regrowth, the surface to be treated is localized to the zones in which baldness (in particular male pattern baldness) appears most often, in particular the crown and the temples.

The composition may be sprayed onto zones of the head that do not have hair.

Preferably, the composition is not a foam.

Preferably, the compressed gas is compressed air. The compressed gas used may thus be non-inflammable, non-fluorinated, non-hydrocarbon, and easily available.

Preferably, the compressed gas used is obtained other than from a liquefied gas. The spray device may thus be different from an aerosol flask having liquefied gas.

The composition for spraying may be stored in the device in the non-pressurized state (i.e. at atmospheric pressure).

In a preferred variant of the invention, the spray device is an airbrush.

Using a device as outlined above, and in particular an airbrush, has numerous advantages.

Compared to application by merely pouring the composition onto the scalp, the device, in particular the airbrush, may provide a greater contact area, a massage sensation, and coolness. In addition, application may be only lightly wetting, without running.

The device, in particular the airbrush, makes it possible to avoid using gloves, while benefiting from satisfactory hygiene as a result of the absence of contact with the scalp.

Compared to certain independent spray devices, the use of an airbrush makes it easy for the user, during use and as desired, to change the operating parameters so as to change the effects that are generated; for example, the user may adjust the flowrate of the jet of composition as a function of the treatment being performed. In addition, the volume of gas (preferably air) that serves as a vector is unlimited when the device, in particular the airbrush, is connected to a compressor. The air is preferably not enriched with oxygen. The composition for spraying may be replenished easily, and different compositions may easily be used successively with the same device, in particular an airbrush. Finally, the user may selectively deliver the vector gas (preferably air) or a mixture of vector gas and composition, if the device, in particular the airbrush, allows it: delivering air may be useful for stimulating the zone before and/or after treatment and/or for accelerating drying of the composition.

The vector gas and the composition for spraying may be mixed inside the device, in particular the airbrush, or outside.

In variant embodiments, the device, in particular the airbrush, may illuminate the zone to be treated so as to assist the user's actions, e.g. it may generate a spot of light showing the axis along which the composition is projected. This assistance may turn out to be useful when the composition for spraying is not colored and when the hair is wet, for example.

The device, in particular the airbrush, may be used to comb the hair, and for this purpose may include a styling endpiece, e.g. situated at the end opposite from the end via which the composition is dispensed, or preferably extending a handle that is used to manipulate the airbrush, in particular when said airbrush is of the pistol type.

The cosmetic composition for spraying may be selected from: anti-dandruff, anti-hairloss, hair-regrowth, stimulating, and protective compositions for the scalp, amongst others. The term “cosmetic composition” should be understood to mean a composition that does not have a therapeutic action, and that may be applied without medical supervision.

Application may be performed without the device, in particular the airbrush, coming into contact with the scalp, except possibly the comb end that is removable for example, so as to enable it to be replaced after cleaning or be selected from a plurality of comb endpieces having characteristics that are different, e.g. concerning their numbers of teeth or their dispositions.

The composition may be applied to hair that is dry or wet. Application may be performed after shampooing, for example.

The vector gas, in particular air, may possibly be heated or cooled before reaching the outlet nozzle of the device, in particular of the airbrush. Heating the vector gas may accelerate drying of the sprayed composition, or may enhance the action of an active agent, e.g. by activating blood circulation locally. Cooling may produce an additional sensation of coolness.

Throughout the specification below, when ranges of values are mentioned, the limits of the ranges are included.

The device, in particular the airbrush, may be held at a distance lying in the range 1 centimeter (cm) to 10 cm from the scalp, preferably 1 cm to 5 cm, while the composition is being sprayed.

The composition may be sprayed after forming a parting in the hair. In order to form such a parting, the user may use a comb or the device, in particular the airbrush, in particular when said device is provided with a comb endpiece.

In an implementation, the method of the invention may include applying a first composition to the scalp and/or the hair, and then applying a second composition to the scalp by means of the device, in particular the airbrush, the second composition being selected as a function of the first or vice versa. The second composition may be applied before the first or vice versa, depending on the treatment being performed.

The first composition, when applied prior to the second, may enhance the action of the second. The two compositions may also react with each other, where appropriate.

In another of its aspects, the invention also provides a treatment system for treating the human scalp, in particular for implementing the above-mentioned method, the system comprising:
a composition for treating the scalp, selected in particular from: anti-dandruff, anti-hairloss, hair-regrowth, anti-seborrheic, anti-inflammatory, anti-irritant or soothing, concealer, stimulating, and protective compositions for the scalp, the composition possibly being cosmetic or dermatological; and
a spray device using a compressed gas and including a reservoir containing said composition. The device is preferably an airbrush, and the compressed gas is preferably compressed air.

The composition may include at least one of the components of the exemplified compositions listed below, for example composition C′, and in particular all active ingredients of the listed compositions.

The term “device including a reservoir” should be understood to mean that the body of the device carries the reservoir or is connected to the reservoir via a flexible hose.

The device, in particular the airbrush, may include an outlet nozzle of diameter lying in the range 0.1 millimeters (mm) to 1 mm, for example, better in the range 0.2 mm to 0.7 mm. The relative pressure of the air at the inlet to the airbrush may lie in the range 0.2 bars to 3 bars, better in the range 0.4 bars to 1 bar. The mean size, i.e. the mean volumic diameter D_{[4,3]}, of the sprayed particles of composition preferably lies in the range 10 micrometers (μm) to 35 μm. The flowrate of composition may be less than or equal to 10 grams per minute (g/min).

The device, in particular the airbrush, may include a comb endpiece. The device, in particular the airbrush, may include a handle, in particular of the pistol type, and the comb endpiece may extend the handle. The comb endpiece may be fastened in removable manner to the device, in particular the airbrush, in particular to the handle.

The vector gas, in particular the air, may be heated or cooled before it leaves the device, in particular the airbrush, e.g. at the outlet from a compressor or a regulator. The temperature on leaving the device, in particular the airbrush, may lie in the range 5° C. to 60° C., for example.

The spray emitted by the device, in particular the airbrush, may be of section that is circular or of some other shape.

The device, in particular the airbrush, may include a control member for controlling the supply of composition, so as to enable the operator to spray the composition selectively. By way of example, the control member includes a surface on which a user's finger presses.

The device, in particular the airbrush, may include a light source for illuminating the scalp, e.g. a laser emitting in the red. The light source may emit substantially on the spraying axis, so as to illuminate the surface to be treated, and may assist in orientating the device, in particular the airbrush.

The light source may be powered by at least one optionally-rechargeable battery that is carried by the device, in particular the airbrush. In a variant, the flexible supply hose may include electrical conductors. The light source may be switched on, depending on whether or not the composition is being sprayed. For example, the control member that opens the supply of composition or that triggers the arrival of vector gas may also act on the operation of the light source.

### Active Ingredients

As mentioned above, the spray device of the invention, in particular the airbrush, may be used in particular to apply a composition for treating the scalp, selected in particular from: anti-dandruff, anti-hairloss, hair-regrowth, anti-seborrheic, anti-inflammatory, anti-irritant or soothing, concealer, stimulating, and protective compositions for the scalp, the composition possibly being cosmetic or dermatological.

The composition may be one or more active agents selected from the compositions below.

The term “anti-seborrheic active agent” means a compound that is capable of controlling the activity of the sebaceous glands.

### Anti-Seborrheic Active Agents

An anti-dandruff active agent that is suitable for the invention may in particular be selected from: retinoic acid; benzoyl peroxide; sulfur; vitamin B6 (or pyridoxine); selenium chloride; sea fennel; mixtures of cinnamon, tea, and octanoylglycine extracts such as Sepicontrol A5 TEA® from the supplier Seppic; mixture of cinnamon, sarcosine, and octanoylglycine, sold in particular by the supplier SEPPIC under the trade name Sepicontrol A5®; zinc salts such as zinc gluconate, zinc pyrrolidonecarboxylate (or zinc pidolate), zinc lactate, zinc aspartate, zinc carboxylate, zinc salicylate, zinc cysteate; copper derivatives and in particular copper pidolate such as Cuivridone® by Solabia; extracts of plants of the species Arnica montana, Cinchona succirubra, Eugenia caryophyllata, Humulus lupulus, Hypericum perforatum, Mentha piperita, Rosmarinus officinalis, Salvia officinalis, and Thymus vulgaris, all sold for example by the supplier MARUZEN; extracts of meadowsweet (Spiraea ulamaria) such as that sold under the trade name Sebonormine® by the company Silab; extracts of Laminaria saccharina (kelp) such as that sold under the trade name Phlorogine® by the supplier Biotechmarine; mixtures of extracts of burnet (Sanguisorba officinalis/Poterium officinale) roots, ginger (Zingiber officinalis) rhizomes, and cinnamon (Cinnamomum cassia) bark such as that sold under the trade name Sebustop® by the supplier Solabia; linseed extracts such as that sold under the trade name Linumine® by the supplier Lucas Meyer; Phellodendron extracts such as those sold under the trade name Phellodendron extract BG by the supplier Maruzen or Oubaku liquid B by the supplier Ichimaru Pharcos; mixtures of argan oil, of Serenoa serrulata (saw palmetto) extract, and of sesame seed extract, such as that sold under the trade name Regu SEB® by the supplier Pentapharm; mixtures of extracts of willow herb, Terminalia chebula, nasturtium and bioavailable zinc (microalgae) such as that sold under the trade name Seborilys® by the supplier Green tech; extracts of Pygeum afrianum such as that sold under the trade name Pygeum afrianum sterolic lipid extract by the supplier Euromed; extracts of Serenoa serrulata such as those sold under the trade name Viapure Sabal by the supplier Actives International, or those sold by the supplier Euromed; mixtures of extracts of plantain, Berberis aquifolium, and sodium salicylate such as that sold under the trade name Seboclear® by the supplier Rahn; clove extract such as that sold under the trade name Clove extract Powder by the supplier Maruzen; argan oil such as that sold under the trade name Lipofructyl® by Laboratoires Serobiologiques; lactic protein filtrates such as that sold under the trade name Normaseb® by the company Sederma; extracts of the alga Laminaria, such as that sold under the trade name Laminarghane® by the supplier Biotechmarine; oligosaccharides of the alga Laminaria digitata such as that sold under the trade name Phycosaccharide AC by the supplier Codif; extracts of cane sugar, such as that sold under the trade name Policasonol® by the supplier Sabinsa; sulfonated schist oil such as that sold under the trade name Ichtyol Pale® by the supplier Ichthyol; meadowsweet (Spiraea ulmaria) extracts such as that sold under the trade name Cytobiol® Ulmaire by the supplier Libiol; sebacic acid, in particular sold in the form of a sodium polyacrylate gel under the trade name Sebosoft® by the supplier Sederma; glucomannans extracted from konjac tuber and modified with alkyl sulfonate chains, such as that sold under the trade name Biopol Beta by the supplier Arch Chemical; extracts of Sophora angustifolia, such as those sold under the trade name Sophora powder or Sophora extract by the supplier Bioland; extracts of Cinchona succirubra bark such as that sold under the trade name Red bark HS by the supplier Alban Muller; extracts of Quillaja saponaria such as that sold under the trade name Panama wood HS by the supplier Alban Muller; glycine grafted onto an undecylenic chain or onto a octanoyl chain, such as that sold under the trade name Lipacide UG OR, Lipacide C₈G by the supplier Seppic; oleanolic acid and nordihydroguaiaretic acid mixture such as that sold in the form of a gel under the trade name AC.Net by the supplier Sederma; phthalimidoperoxyhexanoic acid; (C₁₂-C₁₃)trialkyl citrate sold under the trade name COSMACOL® ECI by the supplier Sasol; (C₁₄-C₁₅)trialkyl citrate sold under the trade name COSMACOL® ECL by the supplier Sasol; 10-hydroxydecanoic acid, and in particular mixtures of 10-hydroxydecanoic acid, sebacic acid, and 1,10-decanediol, such as that sold under the trade name Acnacidol® BG by the supplier Vincience; yeast protein hydrolysates such as ASEBIOL LS by COGNIS (mixed with vitamins); and mixtures thereof.

### Anti-Dandruff Active Agents

The term “anti-dandruff active agent” means a compound that is capable of preventing the appearance of dandruff, of reducing the amount thereof, and/or of making it disappear completely.

An anti-dandruff active agent that is suitable for the invention may in particular be selected from:
derivatives of 1-hydroxy-2-pyridone such as 1-hydroxy-4-methyl-2-pyridone, 1-hydroxy-6-methylpyridone, 1-hydroxy-4,6-dimethyl-2-pyridone, 1-hydroxy-4-methyl-6-(2,4,4-trimethylpentyl)-2-pyridone, 1-hydroxy-4-methyl-6-cyclohexyl-2-pyridone, 1-hydroxy-4-methyl-6-(methyl-cyclohexyl)-2-pyridone, 1-hydroxy-4-methyl-6-(2-bicyclo[2,2,1]heptyl)-2-pyridone, 1-hydroxy-4-methyl-6(4-methylphenyl)-2-pyridone, 1-hydroxy-4-methyl-6[1-[4-nitrophenoxy]-butyl]-2-pyridone, 1-hydroxy-4-methyl-6-(4-cyanophenoxymethyl-2-pyridone), 1-hydroxy-4-methyl-6-(phenylsulfonylmethyl)-2-pyridone, 1-hydroxy-4-methyl-6-(4-bromobenzyl)-2-pyridone and salts thereof; By way of preferred derivative of 1-hydroxy-2-pyridone, mention may be made of the composition sold by HOECHST under the trade name octopyrox (1-hydroxy-4-methyl-6-(2,4,4-trimethylpenthyl)-2-pyridone, monoethanolamine salt;
pyridinethione salts, in particular calcium, magnesium, barium, strontium, zinc, cadmium, tin, and zirconium salts. Zinc pyridinethione salt is particularly preferred. Zinc pyridinethione salt is sold in particular under the trade name zinc omadine by OLIN;
trihalogeno carbamide of formula:

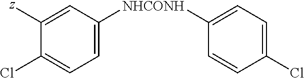

in which Z represents a halogen atom such as chlorine or a C₁-C₄ trihalogenoalkyl group such as CF₃;
triclosan represented by the formula:

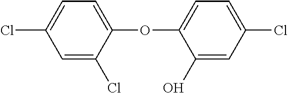

azole compounds such as climbazole, ketoconazole, clotrimazole, econazole, isoconazole, and miconazole;
anti-fungal polymers such as amphotericin B or nystatin;
selenium sulfides, in particular those of formula SₓSe₈₋ₓ, in which x lies in the range 1 to 7;
sulfur in its various forms, cadmium sulfide, allantoin, coal or wood tars, and derivatives thereof, in particular juniper tar oil, salicylic acid, undecylenic acid, fumaric acid, allylamines such as terbinafine;
ellagic acid; and
selenium disulfide.
   Compounds Promoting the Growth of Human Keratinous Fibers and/or Limiting Loss Thereof and/or Promoting an Increase in Their Density

These additional compounds are selected in particular from: lipoxygenase inhibitors as described in EP 0 648 488; bradykinin inhibitors described in particular in EP 0 845 700; prostaglandins and derivatives thereof, in particular those described in. WO 98/33497, WO 95/11003, JP 97-100091, JP 96-134242; agonists or antagonists of prostaglandin receptors; non-prostanoic analogs of prostaglandins as described in EP 1 175 891 and EP 1 175 890, WO 01/74307, WO 01/74313, WO 01/74314, WO 01/74315, or WO 01/72268; and mixtures thereof.

Other additional active compounds promoting the growth of keratinous fibers and/or limiting loss thereof (in particular the hair or the eyelashes) that may be present in the composition of the invention and that may be mentioned are: vasodilators; anti-androgens; cyclosporins and analogs thereof; anti-microbial and anti-fungal agents; anti-inflammatory agents; and retinoids; alone or in a mixture.

Vasodilators that may be used are in particular potassium-channel agonists including minoxidil and compounds described in. U.S. Pat. Nos. 3,382,247, 5,756,092, 5,772,990, 5,760,043, 5,466,694, 5,438,058, 4,973,474, cromakalim, nicorandil, and diaxozide, alone or in combination.

Nicotinic acid esters, including in particular tocopheryl nicotinate, benzyl nicotinate, and C₁-C₆ alkyl nicotinates such as methyl nicotinate or hexyl nicotinate may be used as vasodilators.

Anti-androgens that may be used include, in particular, steroidal or non-steroidal 5α-reductase inhibitors, such as finasteride and the compounds described in U.S. Pat. No. 5,516,779, cyprosterone acetate, azelaic acid and salts and derivatives thereof, and the compounds described in U.S. Pat. No. 5,480,913, flutamide, oxendolone, spironolactone, diethylstilbestrol, and the compounds described in U.S. Pat. Nos. 5,411,981, 5,565,467, and 4,910,226.

The anti-inflammatory agents may be selected from: steroidal anti-inflammatory agents such as glucocorticoids, corticosteroids (e.g. hydrocortisone); and non-steroidal anti-inflammatory agents such as glycyrrhetinic acid and α-bisabolol, benzydamine, salicylic acid, and the compounds described in EP 0 770 399, WO 94/06434, and FR 2 268 523.

Retinoids may be selected from retinoic acid, isotretinoin, acitretin, tazarotene, retinal, and adapalene.

Other active compounds for promoting growth and/or limiting loss of keratinous fibers such as the hair and the eyelashes that may be mentioned are: aminexil; 6-0-[(9Z,12Z)-octadeca-9,12-dienoyl]hexapyranose; benzalkonium chloride; benzethonium chloride; phenol; oestradiol; chlorpheniramine maleate; chlorophylline derivatives; cholesterol; cysteine; methionine; arginine; menthol; peppermint oil; calcium pantothenate; panthenol; resorcinol; protein kinase C activators; glycosidase inhibitors; glycosaminoglycanase inhibitors; pyroglutamic acid esters; hexosaccharidic or acylhexosaccharidic acids; substituted arylethylenes; N-acylamino acids; flavonoids; ascomycin derivatives and analogs; histamine antagonists; saponins; proteoglycanase inhibitors; oestrogen agonists and antagonists; pseudoterines; cytokines and growth factor promoters; inhibitors of IL-1 or of IL-6; IL-10 promoters; TNF inhibitors; benzophenones and hydantoin; vitamins such as vitamin D, vitamin B12 analogs, panthenol, vitamin B8; triterpenes such as ursolic acid and the compounds described in U.S. Pat. No. 5,529,769, U.S. Pat. No. 5,468,888, U.S. Pat. No. 5,631,282; anti-pruriginous agents such as thenaldine, trimeprazine, or cyproheptadine; anti-parasitic agents, in particular metronidazole, crotamiton, or pyrethrinoids; calcium antagonist agents such as cinnarizine, diltiazem, nimodipine, verapamil, alverine, and nifedipine; hormones such as estriol or analogs thereof, thyroxine and salts thereof, progesterone; FP receptor (type F prostaglandin receptor) antagonists such as latanoprost, (5E)-7-{(1R,2R,3R,5S)-3,5-dihydroxy-2-[(3R)-3-hydroxy-5-phenyl pentyl]cyclopentyl}hept-5-enoic acid, bimatoprost, travoprost, unoprostone, and butaprost; O-acyl derivatives obtained by partial or total esterification of vitamin F by glucose, as described in application EP 1 688 128; 15-hydroxy prostaglandin dehydrogenase inhibitors; and mixtures thereof.

Other active compounds for promoting growth and/or limiting loss of keratinous fibers such as the hair and the eyelashes, that may be used in combination with the compound of formula (I) and that may be mentioned are: pyridine dicarboxylate derivatives or a salt thereof such as those described in application EP 1 352 629 and more particularly diethyl pyridine-2,4-dicarboxylate.

Other than the above-mentioned anti-inflammatory agents, anti-irritant/soothing active agents that may be mentioned are: strontium salts, β-glycyrrhetinic acid, glycyrrhizic acid, azulene, essential oils, extracts of chamomile, oats, aloe vera, verbena, tilia, and licorice.

As should be apparent, the invention can provide a number of advantageous features and benefits. It is to be understood that, in practicing the invention, an embodiment can be constructed to include one or more features or benefits of embodiments disclosed herein, but not others. Accordingly, it is to be understood that the preferred embodiments discussed herein are provided as examples and are not to be construed as limiting, particular since embodiments can be formed to practice the invention that do not include each of the features of the disclosed examples.

### BRIEF DESCRIPTION OF THE DRAWINGS

A better understanding of the invention will be gained from reading the following description in conjunction with the accompanying figures. The figures are offered purely as a guide and by way of example, and in no way limit the invention.

The invention can be better understood on reading the following detailed description of non-limiting embodiments thereof, and on examining the accompanying drawings, in which:

FIG. 1 is a diagrammatic and fragmentary view showing an embodiment of a treatment system that is suitable for being used in accordance with the invention; and

FIG. 2 shows an example of an airbrush.

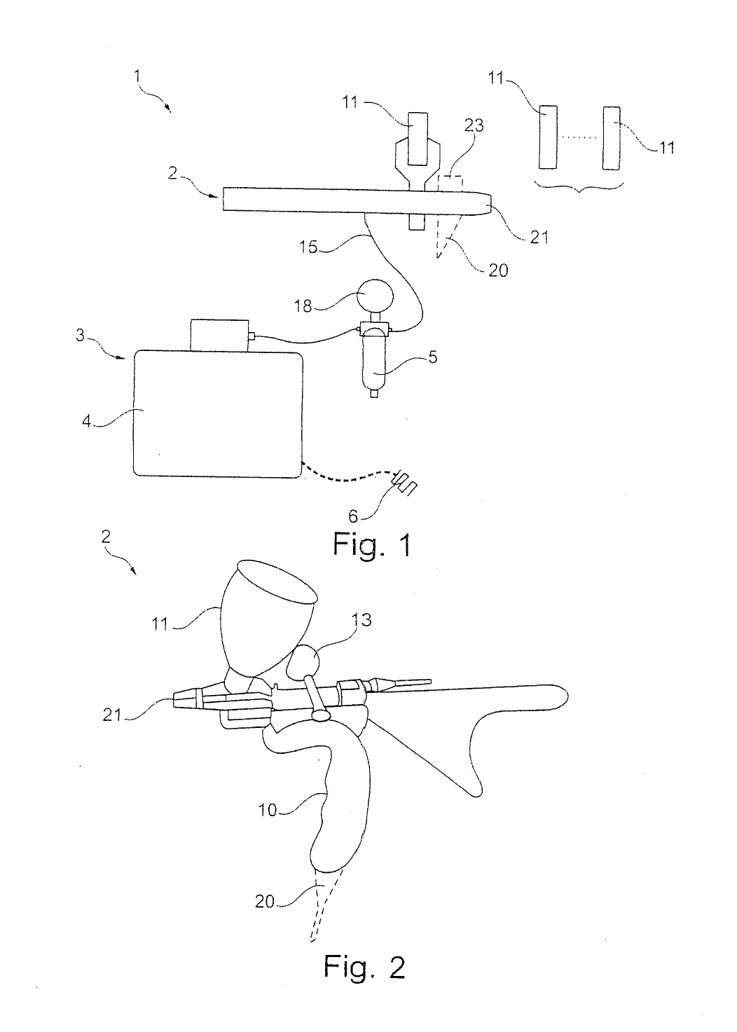

FIG. 1 shows an embodiment of a treatment system 1 that is suitable for being used to implement the treatment method of the invention.

### DETAILED DESCRIPTION OF THE PREFERRED EMBODIMENTS

Referring now to the drawings, like reference numerals are utilized to designate identical or corresponding parts throughout the several views.

As shown, the treatment system 1 may comprise an airbrush 2 that is connected to a compressed-gas source, e.g. comprising an air compressor 4 that is connected in conventional manner to the airbrush 2 via a pressure regulator 5 and a flexible hose 15. The compressed-gas source may equally well be an interchangeable or rechargeable capsule of compressed gas.

The operation of the compressor 4 may possibly be controlled by a foot-actuated system 6 or by any other control means, e.g. hand-controlled or voice-controlled means. In variants, the operation of the compressor may equally well be triggered automatically by detecting movement of the airbrush or its removal from a stand.

The compressor 4 preferably emits sound that is quieter than 40 decibels (dB) and preferably provides an air flow that is greater than or equal to 15 liters per minute (L/min), the compressor optionally has an air supply, preferably with thermal protection, and also preferably has an outlet that is fitted with a quick coupler for fastening the connection hose to the airbrush.

The compressor 4 may be a single-piston, dual-piston, dry, or oil-bath compressor, and the pressure regulator 5 is preferably fitted with a pressure gauge 18.

In known manner, the airbrush 2 includes a handle portion that, by way of example, is defined by the body of the airbrush when said airbrush presents the shape of a pen, or by a handle 10 when said airbrush is of the pistol type, as shown in FIG. 2.

The airbrush 2 may carry a reservoir 11 containing the composition for spraying, said reservoir being in the form of a removable cup for example, that is possibly rechargeable, and that may be situated at the top portion of the airbrush so as to enable the composition contained in said cup to flow, by gravity, into a dispenser channel in which the composition is mixed with a vector gas before being sprayed.

The reservoir 11 may be provided with graduations so as to enable the user to see more easily the quantity of composition that is available. The volume of composition contained in the reservoir 11 lies in the range 1 milliliter (mL) to 100 mL, for example.

The airbrush 2 may include any trigger means for triggering spraying, e.g. in the form of a control member such as a lever 13, as shown in FIG. 2, that is suitable for being actuated using a finger of the user's hand that is holding the airbrush.

Examples of airbrushes that are suitable for being used in the invention and that may be mentioned are the airbrush referenced A 4700 from the supplier Azteck, the airbrush referenced Kustom micron CM from the supplier Iwata, and the airbrush referenced Evolution infiniti 2 in 1 from the supplier Harder and Steenbeck, those airbrushes being of the type held like a pen, with a reservoir in the form of a cup.

The airbrush referenced Kustom revolution TR from Iwata, or the airbrush referenced Coleni® from Harder and Steenbeck may equally well be used, the Coleni® model being preferred for its ease of handling that is particularly suited to treating the scalp, and being the model shown in FIG. 2.

Preferably, the flexible hose 15 that connects the airbrush to the compressor, and in particular to the air pressure regulator 5, presents a length that is less than or equal to 5 meters (m), and its inside diameter is equal to 4 mm, for example. Preferably, the flexible hose 15 is provided with quick-coupler endpieces.

The airbrush 2 may advantageously include a comb endpiece 20 that may enable the user who holds the airbrush to move the hair out of the away during treatment so as to expose the scalp, in particular to form a parting in the hair. By way of example, the comb endpiece 20 extends the handle 10 of an airbrush of the pistol type, as shown in FIG. 2. It may equally well be fastened elsewhere, e.g. it may be situated at the rear end of the airbrush, i.e. the end remote from the nozzle 21 for projecting the composition, or it may be situated on the side other than at a handle, as shown diagrammatically in FIG. 1.

The airbrush 2 may possibly include a lighting system 23 for illuminating the zone towards which the composition is projected. By way of example, the lighting system 23 comprises one or more light-emitting diodes emitting light that is white or of some other color. Where appropriate, the angle of divergence of the light beam emitted by the light source 23 may be selected so that the illuminated area corresponds substantially to the area touched by the composition when projected from a predefined working distance.

The light source 23 may equally well include a laser pointer making it possible to project a spot of light or a target onto the zone to be treated, making it easier for the user to direct the airbrush in the correct direction. This makes it possible to direct the composition as well as possible onto the scalp, reducing losses due to spraying onto the hair.

Where appropriate, the projected target appears blurred when the airbrush is not at the correct spraying distance.

The light source 23 may also seek to perform a light-therapy treatment on the scalp, e.g. by emitting at one or more wavelengths selected to exert a beneficial effect on hair regrowth, in particular a wavelength in the red.

The airbrush 2 may be made available to the user with a plurality of additional reservoirs 11, as shown in FIG. 1, e.g. each containing the same composition as the composition present in the reservoir that is in place on the airbrush, so as to enable the user to replace an empty reservoir quickly with a full reservoir. The additional reservoirs may be provided with a quick connector, automatically closing when the reservoir is withdrawn from the airbrush and automatically opening when the reservoir is put into place on the airbrush. The reservoirs 11 may also comprise closing means such as a plug, perforated by opening means such as a needle, present on the airbrush. The additional reservoirs may equally well contain different compositions, seeking to perform different treatments, and the user may choose between the reservoirs, the reservoir with the composition that corresponds to the treatment to be performed.

The spraying parameters, in particular the flowrate of the vector gas (preferably air) and/or the flowrate of sprayed composition, may be adapted manually by the user each time the reservoir 11 is changed, when that is necessary, or during use.

In a variant, the treatment system 1 is arranged to adapt the operating parameters automatically, as a function of the reservoir that is in place and of the composition that is contained therein, e.g. by means of the treatment system, e.g. the airbrush 2, recognizing which reservoir is being used. By way of example, the reservoirs containing different compositions may present identifiers that are recognized by the treatment system. For example, the different reservoirs may include an electronic chip, an optical code, or portions in relief that are detected by a suitable detector, e.g. present on the airbrush 2, with a processor making it possible to control at least one actuator, so as to change an operating parameter as a function of the information read.

The compressed-air pressure at the inlet to the airbrush 2 may lie in the range 0.2 bars to 3 bars, e.g. about 0.6 bars.

Preferably, the nozzle 21 equipping the airbrush is selected so that the mean size of the sprayed droplets of composition is centered on a value lying in the range 10 μm to 35 μm, e.g. about 23 μm (size measured at a distance of 15 cm from the outlet of the nozzle).

The choice of diameter of the nozzle makes it possible to act on the particle size of the spray and on its divergence. By way of example, a nozzle of 0.2 mm in diameter may be used, in particular on the airbrush referenced Colani, thereby producing a jet that is accurate and that can be felt by the user. A nozzle diameter of 0.2 mm is suitable most particularly when used together with a pressure of 0.8 bars at the inlet of the airbrush, so as to obtain a flowrate of composition of about 3.5 g/min. For the Kustom micron CM airbrush, the nozzle diameter is 0.5 mm and the inlet pressure is 0.4 bars with a flowrate of composition of about 6 g/min. Preferably, care should be taken to ensure that the content of particles of size that is less than 10 μm is less than 20%, and the content of particles of size that is less than 5 μm is less than 5%.

The treatment system 1 may be used on a dry or damp scalp in order to spray thereon treatment compositions having in particular anti-dandruff, anti-hairloss, hair-regrowth, stimulating, anti-seborrheic, or anti-inflammatory properties, amongst other applications.

In implementations of the invention, the composition is sprayed with the airbrush in combination with applying a conventional treatment, e.g. shampooing prior to treatment by the airbrush, conditioning, or a hair care mask. All of the conventional treatments may be followed by spraying a scalp-treatment composition onto the dry or damp scalp, in particular an anti-dandruff, anti-hairloss, hair-regrowth, anti-seborrheic, or anti-inflammatory composition.

In other implementations of the invention, a stimulating oil having anti-greasy properties and a washing foam are applied to the hair and the scalp, and the scalp may then be treated by an anti-hairloss composition that is sprayed with the airbrush, the anti-hairloss composition being selected as a function of the nature of the previously-applied composition.

The use of the airbrush may also be followed by a conventional treatment such as a permanent wave, a relaxing treatment, bleaching, color stripping, or coloring.

By way of example, it is possible to apply, by means of the airbrush, a composition for protecting the scalp, in particular an anti-irritant/soothing composition and/or concealers, before performing a permanent-wave, relaxing, bleaching, or color-stripping treatment.

A few examples are given below of composition formulations that are suitable for being sprayed by means of the airbrush. The proportions indicated are by weight.

While using the airbrush, the operator may hold said airbrush with one hand, and use a comb with the other hand to clear the scalp at locations for treatment.

The application of the composition by means of the airbrush may be followed by a massage, before or after the composition is sprayed. By way of example, the massage may be performed with the fingers or using a massage device that is brought into contact with the scalp, e.g. a vibrator device.

In implementations, a composition may be applied with or without the airbrush, then, by means of the airbrush, blown with hot air onto the zone for treatment, e.g. so as to activate an active agent contained in the composition, e.g. an anti-hairloss active agent. Raising the temperature may accelerate blood circulation locally and may enhance the action of the active agent. The hot air at the outlet from the airbrush may thus be at a temperature lying in the range 30° C. to 60° C.

Whatever the composition applied, preferably while using the airbrush, the outlet nozzle is maintained at a distance from the scalp lying in the range 1 cm to 10 cm, e.g. in the range 1 cm to 5 cm.

### EXAMPLES

### A Single-Treatment Method

| | | Composition for spraying with |
|---|---|---|
| Method | Scalp | the airbrush |
| 1 | Dry or damp | Anti-dandruff composition |
| | | Anti-hairloss composition |
| | | Hair-regrowth composition |
| | | Anti-seborrheic composition |
| | | Anti-inflammatory composition, . . . |

A Treatment Method Combined with a Conventional Treatment

| | | Composition for spraying with |
|---|---|---|
| | Conventional | the airbrush |
| Methods | treatments | Hair and scalp dry or damp |
| 2 | Shampooing | Formulas for treating the scalp: |
| 3 | Conditioning | Anti-dandruff composition |
| 4 | Shampooing + | Anti-hairloss composition |
| | conditioning | Hair-regrowth composition |
| 5 | Hair care mask | Anti-seborrheic composition |
| | | Anti-inflammatory composition, . . . |
| | | Composition for spraying |
| Method | Pre-treatment | with the airbrush |
| 6 | Anti-greasy | Anti-hairloss composition |
| | stimulating oil then | formulated as a function |
| | washing foam | of the pre-treatment. |
| Methods | Pre-treatment by airbrush | Final treatment |
| 7 | Formulas for protecting the | Permanent wave |
| 8 | scalp: | Relaxing |
| 9 | Anti-irritants | Bleaching |
| 10 | Concealers | Color stripping |

### Hair-Regrowth Formula A:

| | A | Possible methods |
|---|---|---|
| Safflower glucoside | 3 | 1, 2, 3, 4, and 5 |
| Ethanol | 50 | |
| Water | 47 | |

### Anti-Hairloss Formulae B and C:

| | B | C | Possible methods |
|---|---|---|---|
| Diaminopyrimidine | 0.2 | 1.5 | 1, 2, 3, 4, 5, and 6 |
| oxide | | | |
| Safflower glucoside | — | 0.1 | |
| PEG-40 Hydrogenated | — | 0.1 | |
| castor oil | | | |
| Ethanol | 48 | 50.7 | |
| Water | 51.8 | 47.6 | |

### Anti-Hairloss Formula C′ (May be Used Instead of Formulae B or C)

| | |
|---|---|
| L-Arginin | 1.5% |
| Calcium pentothenate | 0.1% |
| Nicotinic Acid | 0.1% |
| DLalpha-tocopheryl acetate | 0.05% |
| PEG-40 hydrogenated castor oil | 1% |
| L-menthol | 0.1% |
| Ethanol | 35% |
| Water | 62.15% |

### Anti-Dandruff Formulae D to I:

| | | | | | | | Possible |
|---|---|---|---|---|---|---|---|
| | D | E | F | G | H | I | methods |
| Piroctone | 0.2 | — | — | — | — | — | 1, 2, 3, 4, |
| olamine | | | | | | | and 5 |
| (octopirox) | | | | | | | |
| Zinc | 0.2 | — | — | — | — | — | |
| pyrithione | | | | | | | |
| Selenium | — | 0.5 | — | — | — | — | |
| sulfide | | | | | | | |
| Ellagic | — | — | 0.5 | — | — | — | |
| acid | | | | | | | |
| Fragrance | 0.2 | — | — | — | — | — | |
| Ethanol | 40.8 | 41.5 | 18.5 | — | — | 36.1 | |
| Water | 58.8 | 58 | 81 | 80 | 80 | 62.9 | |

### Anti-Seborrheic Formulae H and I:

| | H | I | Possible methods |
|---|---|---|---|
| Zinc Pyrrolidone | 1 | — | 1, 2, 3, 4, and 5 |
| carboxylate | | | |
| Hydrolyze yeast | | 0.5 | |
| protein (ASEBIOL LS) | | | |
| Ammonium | | 0.2 | |
| glycyrrhizate | | | |
| Ethanol | — | 40 | |
| Water | 99 | 59.3 | |

### A Treatment Method Using the Anti-Hairloss Formula C for Implementing Method 6

| | |
|---|---|
| 1. | Treating the scalp with a stimulating oil |
| | (e.g. formula J). |
| 2. | Wetting with water. |
| 3. | Treating with a foam (e.g. formula K). |
| 4. | Rinsing. |
| 5. | Optionally drying (hair dryer). |
| 6. | Applying 6 millimeters of anti-hairloss formula C |
| | with the airbrush (e.g. Colani ®). |
| | Adjusting the compressed-air pressure in the range |
| | 0.5 bars to 0.8 bars as a function of the massage |
| | effect desired. |
| | 0.2 mm nozzle, and maximum airflow of the jet: |
| | 3.4 g/min. |
| 7. | The applied solution, do not rinse, but style. |

### Foam Formula K

| | |
|---|---|
| OE 60 hydrogenated castor oil | 0.25 |
| Permulen TR2 | 0.15 |
| Glycerine | 4.0 |
| Ethanol | 13.0 |
| PEG 8 | 0 or 0.10% |
| Xanthan gum | 0.2 |
| Lactic acid | 0.2 |
| Triethanolamine | 0.6 |
| Dimethylether | 20.0 |
| Isobutene/propane/butane mixture | 15.0 |
| Preservatives | qs |
| Water | qsp 100 g |

### Stimulating Oil J

| | |
|---|---|
| Noctanoyl glycine | 0.5 |
| Quaternium 80 | 0.4 MA |
| Polysorbate 80 | 1.5 |
| Cetyl methyl ammonium chloride | 0.5 |
| Merquat 100 | 1 MA |
| Glycerine | 1 |
| Hydroxyethyl cellulose | 0.4 g |
| preservative | qs |
| Water | qsp 100 g |

### Comparative Example

The anti-hairloss formula was poured directly onto the scalp from a flask provided with a teat, starting from step 6 of the preceeding example.

| | Anti-hairloss care product |
|---|---|
| Anti-hairloss care product | applied with the airbrush |
| applied conventionally | (e.g. Colani ®) |
| Application by pouring or | Application by pulling (or |
| by pressing a teat. | pressing) on the trigger |
| It is necessary to stop | for delivering composition. |
| regularly so as to avoid | It is possible to move |
| soaking any given zone. | the tool over the surface |
| Difficult to maintain the | of the head without |
| action while moving the | stopping the jet. |
| flow over the surface of | Running is avoided. |
| the head (parting by | Application extremely |
| parting) | precise parting by parting. |
| The zone for applying the | The treated zone is |
| composition is not precise. | almost dry and it is |
| Massage with the fingers | possible to apply again if |
| so as to ensure the formula | necessary. |
| is distributed. | It is not necessary to |
| | distribute the composition |
| | with the fingers. |

Naturally, the invention is not limited to the examples described above.

When the airbrush emits only vector gas, in particular air, it is possible in particular to use the treatment system in order to dry the composition applied beforehand or to stimulate its action.

A single compressor may be used with a plurality of airbrushes, e.g. the compressor being situated outside the room in which the treatment is performed so as to reduce noise nuisance.

A reservoir carried by the airbrush may be replaced by a reservoir connected to the airbrush via a duct, the composition being sucked into the airbrush by the suction created by the Venturi effect by the vector gas flowing through the airbrush. A pump may equally well be used to bring the composition for spraying to the airbrush.

Preferably, the spray is circular, but various nozzles may be used so as to have sprays that are flat or that have some other shape.

The compressor may be replaced by a supply of compressed air, e.g. compressed air in a cylinder.

Spray devices other than an airbrush may be used, e.g. devices in which a compressed-gas source is incorporated in the device and is manipulated with said device during use. By way of example, the device may be as described in publication EP 1 355 613 B1 or EP 1 470 867 A2.

The expression “comprising a” should be understood as being synonymous with “comprising at least one” unless specified to the contrary.

Obviously, numerous modifications and variations of the present invention are possible in light of the above teachings. It is therefore to be understood that within the scope of the appended claims, the invention may be practiced otherwise than as specifically described therein.

1. A cosmetic treatment method for treating the human scalp comprising:
applying a cosmetic composition and/or a gas to the scalp with a spray device operating with a compressed gas,
wherein a mean size of the sprayed particles of composition lying in a range 10 μm to 35 μm.

2. The method according to claim 1, wherein the compressed gas is compressed air.

3. The method according to claim 1, wherein the compressed gas is produced by a compressor or is dispensed from a capsule that is interchangeable or rechargeable, the compressor or the capsule configured to be connected to the spray device.

4. The method according to claim 1, wherein the spray device is an airbrush.

5. The method according to claim 4, wherein the applying a cosmetic composition is performed without the airbrush coming into contact with the scalp.

6. The method according to claim 1, further comprising heating the air before the air leaves the spray device.

7. The method according to claim 1, further comprising spraying the composition with a flowrate that is less than 10 g/min.

8. The method according to claim 1, the composition being other than a foam.

9. The method according to claim 1, the cosmetic composition comprising a composition for treating the scalp, selected from: anti-dandruff, anti-hairloss, hair-regrowth, anti-seborrheic, anti-inflammatory, anti-irritant or soothing, concealer, stimulating, and protective compositions for the scalp.

10. A treatment system for treating the human scalp, comprising:
an airbrush including a reservoir configured to contain a composition for treating the scalp, selected from: anti-dandruff, anti-hairloss, hair-regrowth, anti-seborrheic, anti-inflammatory, anti-irritant/soothing, and concealer compositions; and
a plurality of additional reservoirs containing a same composition as the composition present in the reservoir that is in place on the airbrush.

11. The system according to claim 10, wherein the airbrush includes an outlet nozzle having a diameter in the range of 0.1 mm to 1 mm.

12. The system according to claim 11, wherein the diameter is in the range 0.2 mm to 0.7 mm.

13. The system according to claim 11, wherein a pressure of the air is in the range 0.2 bars to 3 bars.

14. The system according to claim 10, the airbrush including a comb endpiece and/or a light source that illuminates the scalp.

15. The system according to claim 14, wherein the comb endpiece is attached to a handle of the air brush.

16. The system according to claim 15, wherein the comb endpiece is situated at a lower end of the handle.

17. The system according to claim 11, wherein the air is configured to be heated or cooled before it leaves the airbrush.

18. The system according to claim 11, wherein the flowrate of composition during spraying is less than 10 g/min.
